(19) United States
(12) Patent Application Publication
(10) Pub. No.: US 2014/0271850 A1
(43) Pub. Date: Sep. 18, 2014

(54) Controlled-Release Pharmaceutical Compositions Comprising Lamotrigine and Methods of Producing Same

(71) Applicant: PharmTak, Inc., San Jose, CA (US)

(72) Inventors: I-Lan Tung Sue, San Jose, CA (US); Jung-Chung Lee, Saratoga, CA (US)

(73) Assignee: PHARMTAK, INC., San Jose, CA (US)

(21) Appl. No.: 14/210,039

(22) Filed: Mar. 13, 2014

Related U.S. Application Data
(60) Provisional application No. 61/781,344, filed on Mar. 14, 2013.

(51) Int. Cl. A61K 9/28; A61K 31/53

(52) U.S. Cl. CPC A61K 9/2833; A61K 31/53
USPC 424/472; 514/242

(57) ABSTRACT

Described herein is a pharmaceutical composition comprising lamotrigine as an active ingredient providing a prolonged release characteristic to allow once a day dosage regime. The composition comprises lamotrigine and a swelling agent forming a core or an extended-release composition. The core or an extended-release composition may be coated with a hydrophobic, pH-independent polymer to provide a modified extended-release composition. The formulation may comprise other pharmaceutically acceptable excipients that may act as additional release rate modifier(s), e.g., a pore forming agent in the hydrophobic polymer coat/film. Also described are the processes of preparing such dosage forms and their use.

### CROSS-REFERENCE TO RELATED APPLICATIONS

This application claims the benefit of U.S. Provisional Application No. 61/781,344, filed on Mar. 14, 2013, which is incorporated by reference herein in its entirety.

### TECHNICAL FIELD

This invention relates to controlled-release dosage forms of lamotrigine including the pharmaceutical composition and methods for producing them.

### BACKGROUND

Lamotrigine is an antiepileptic drug (AED) of the phenyl triazine class. Its chemical name is 6-(2,3-dichlorophenyl)-1,2,4-triazine-3,5-diamine (or 3,5-diamino-6-(2,3-dichlorophenyl)-1,2,4-triazine). It has the following structural formula:

[chemical structure]

Oral immediate release and chewable dispersible tablet formulations of lamotrigine are commercially available in the United States of America under the brand names Lamictal® and Lamictal® CD, respectively, from GlaxoSmithKline, USA. Lamotrigine is rapidly and completely absorbed after oral administration with negligible first-pass metabolism (absolute bioavailability is 98%). To ameliorate the drawbacks associated with conventional immediate release formulations, lamotrigine is formulated into a controlled release once-daily dosage form which is available in the USA, as Lamictal® XR extended release tablets from GlaxoSmithKline Inc. Lamotrigine prescribing information has a black box warning about life-threatening skin reactions, including Stevens-Johnson syndrome, Drug Reaction (or Rash) with Eosinophilia and Systemic Symptoms (DRESS syndrome) and toxic epidermal necrolysis.

Adverse events associated with lamotrigine are typical of antiepileptic drugs, namely dizziness, ataxia, diplopia, somnolence, headache, and asthenia.

Neurological side effects are normally seen at higher plasma concentrations (which are most likely to occur at peak plasma concentrations).

In the treatment of epilepsy it is speculated that the troughs may lead to breakthrough seizures and the peak plasma concentration may result in some adverse events (AE) occurring in some patients or alternatively the rate of increase in plasma concentration in the initial stages before the peak plasma concentration is achieved may also affect the AE profile.

There are suggestions, yet to be proven, that the risk of side-effects, severe or otherwise, may be increased if you take Lamictal® XR while taking valproate [Depakene® (valproic acid) or Depakote® (divalproex sodium)], take a higher starting dose of Lamictal® XR than your healthcare provider prescribed, or increase your dose of Lamictal® XR faster than prescribed. Thus, it may be beneficial to have a formulation that provides a delayed onset of active agent release or slower release of the active agent from the dosage form or both.

In the present case, the inventors have prepared extended-release and/or modified extended-release formulations of lamotrigine using techniques that formulate a dosage form which manifests these desired characteristics and is patient compliant, can be easily manufactured, and will hopefully provide for fewer side-effects.

The present invention can reduce these side effects by controlling the plasma levels of lamotrigine through the use of an extended-release and/or modified extended-release formulation of lamotrigine. The formulations disclosed herein may help maintain the steady state concentration with little fluctuations. The reduced incidence of these neurological side effects improves patient compliance with their prescribed therapy.

### BRIEF SUMMARY OF THE INVENTION

Described herein are controlled-release composition of an active agent, e.g., lamotrigine, and methods of manufacturing the composition(s). Disclosed herein are controlled-release compositions comprising:

(a) an extended-release core comprising:
(i) lamotrigine;
(ii) a swelling agent; and
(iii) optionally, one or more pharmaceutically acceptable excipients; and

(b) optionally, a coating agent comprising:
(i) a hydrophobic, pH-independent polymer; and
(ii) optionally, a pore forming agent.

In an aspect, a modified extended-release composition comprises:

(a) an extended-release core comprising:
(i) lamotrigine;
(ii) a swelling agent; and
(iii) optionally, one or more pharmaceutically acceptable excipients; and

(b) a coating agent comprising:
(i) a hydrophobic, pH-independent polymer; and
(ii) optionally, a pore forming agent.

In an aspect, an extended-release composition comprises an active agent or its pharmaceutically acceptable salts, polymorphs, solvates, hydrates, derivatives thereof, and one or more swelling agent.

In an aspect, an extended-release composition comprises an active agent or its pharmaceutically acceptable salts, polymorphs, solvates, hydrates, derivatives thereof, at least one pharmaceutically acceptable excipient, and one or more swelling agent.

In an aspect, a modified extended-release composition comprises lamotrigine or its pharmaceutically acceptable salts, polymorphs, solvates, hydrates, derivatives thereof, one or more swelling agents, and a coating agent.

In some embodiments, an extended-release composition may serve as the core for a modified extended-release composition. In certain embodiments, an extended-release composition is coated with one or more hydrophobic, pH-independent polymers.

In an aspect, a method of manufacturing (or producing) a controlled-release composition comprises:
(a) granulating lamotrigine and one or more swelling agent, e.g., hydrophilic polymer, to form granules and/or agglomerates;
(b) blending the granules and/or agglomerates of step (a) with one or more pharmaceutically acceptable excipients to form a dry blend; and
(c) preparing a dosage form from the dry blend.

In another aspect, a method of manufacturing (or producing) a controlled-release composition comprises:
(a) granulating lamotrigine, one or more swelling agent, e.g., hydrophilic polymer, and one or more pharmaceutically acceptable excipients to form granules and/or agglomerates;
(b) blending the granules and/or agglomerates of step (a) with one or more lubricant to form a dry blend; and
(c) preparing a dosage form from the dry blend.

In another aspect, a method of manufacturing (or producing) a controlled-release composition comprises:
(a) granulating lamotrigine, one or more swelling agent, e.g., hydrophilic polymer, to form granules and/or agglomerates;
(b) blending the granules and/or agglomerates of step (a) with one or more pharmaceutically acceptable excipient to
(c) blending a lubricant with the dry blend to form a lubricated dry blend; and
(c) preparing a dosage form from the dry blend.

In another aspect, a method of manufacturing (or producing) a controlled-release composition comprises:
(a) granulating lamotrigine and one or more swelling agent, e.g., hydrophilic polymer, to form granules and/or agglomerates; and
(b) preparing a dosage form from the granules and/or agglomerates.

In some embodiments of the manufacturing methods disclosed herein, the manufacturing method further includes coating the dosage form with one or more coating agent(s), for example, to prepare a modified extended-release composition. Functional and/or non-functional coating agent(s) may be used. In some embodiments, the coating agent is functional. In some embodiments, the coating agent is non-functional. In some embodiments, the coating agent is a hydrophobic, pH-independent polymer. In some embodiments, the coating agent further includes one or more pore former(s).

In all embodiments, the active agent is lamotrigine. In some embodiments, the lamotrigine is present, for example, in a dosage form, in an amount of about 25 mg to about 400 mg. In some embodiments, the lamotrigine content is selected from 25, 50, 100, 200, 300 and 400 mg. In embodiments, the lamotrigine is present in an amount from about 5% w/w to about 70% w/w. In a specific embodiment, lamotrigine content is about 31% w/w. In some embodiments, the lamotrigine content is about 6% to about 70% w/w. In various embodiments, the lamotrigine content is about 8%, 17%, 33% 50% or 60% w/w.

In an embodiment, the extended-release composition comprises about 5% w/w to about 70% w/w of lamotrigine, or about 6% w/w to about 70% w/w lamotrigine, from about 5% w/w to about 80% w/w swelling agent(s) having a viscosity s10,000 cps, from about 0% w/w to about 70% w/w lactose, or about 0% w/w to about 40% w/w lactose, and from about 0.1% w/w to about 5% w/w magnesium stearate, or about 0.5% w/w to about 4% w/w magnesium stearate.

In a specific embodiment, the extended-release composition comprises about 5% w/w to about 70% w/w of lamotrigine (or about 6% w/w to about 70% w/w, or about 6% w/w, 8% w/w, 17% w/w, 33% w/w, 50% w/w, or 60% w/w lamotrigine), from about 5% w/w to about 80% w/w Hypromellose 2208 (or about 10% w/w to about 40% w/w, about 20% w/w to about 30% w/w, or about 25% w/w Hypromellose 2208), from about 0% w/w to about 80% w/w Hypromellose 2910 (or about 0% w/w to about 70% w/w, about 2% w/w to about 25% w/w, about 5% w/w to about 15% w/w, or about 10% w/w Hypromellose 2901), from about 0% w/w to about 70% w/w lactose (or about 0% w/w to about 70% w/w, about 5% w/w to about 60% w/w, about 10% w/w to about 15% w/w, about 2% w/w to about 11% w/w, or about 0% w/w to about 40% w/w lactose), from about 0% w/w to about 70% w/w microcrystalline cellulose (or about 0% w/w to about 60% w/w, about 5% w/w to about 60% w/w, or about 15% w/w to about 50% w/w microcrystalline cellulose), and from about 0.1% w/w to about 5% w/w magnesium stearate (or about 0.3% w/w to about 3% w/w, about 0.5% w/w to about 4% w/w, about 2% w/w to about 3% w/w, or about 1% w/w magnesium stearate).

In some embodiments, dosage forms of the extended-release and modified extended-release compositions described herein are in the form of tablets, capsules, pills, mini-tabs, sprinkles, pellets, or beads, or any other form that is suitable for administration to an individual in need thereof. In some embodiments, the dosage form is free-flowing granules or sachets. In some embodiments, the dosage form is an extended-release composition. IN some embodiments, the dosage form is prepared by compression.

In all embodiments, the at least one swelling agent is one or more swellable polymer, or one or more hydrophilic polymer, or a mixture of hydrophilic polymers, or a mixture of swellable polymers, or a mixture of hydrophilic polymers and swellable polymers. The swelling agents may be selected from Hypromellose, microcrystalline cellulose, carboxymethylamide, cross-linked polyvinylpyrrolidone, polyvinylacetate, polyvinylalcohols, polyoxyethylene glycols, non-crosslinked polyvinylpyrrolidone, cellulose derivatives (such as hydroxyethyl cellulose, hydroxypropyl cellulose, hydroxypropyl methylcellulose, methylcellulose, carboxymethyl cellulose and the like), ethylcellulose resins (Ethocel®), cellulose powder, noncellulose polysaccharides (galactomannans, guar gum, carob gum, gum arabic, sterculia gum, agar, alginates and the like), xanthan gum, carrageenan, tragacanth, casein, zein, gelatin, polyethylene oxide (Polyox resins), sodium carboxymethyl cellulose, calcium carboxymethylcellulose, croscarmellose sodium, vinylpyrrolidone/vinyl acetate copolymer (for example marketed as Plasdone® S-630), and polyethylene glycol. The swelling agent(s) is present in an amount of about 5% to about 80% by weight. In a specific embodiment, the swelling agent is a mixture of Hypromellose 2208, Hypromellose 2910 and microcrystalline cellulose. In some embodiments, the at least one swelling agent forms a matrix.

In all embodiments in which at least one excipient is included, the at least one pharmaceutically acceptable excipient is selected from diluents, fillers, binders, flow enhancers, compression enhancers, stabilizers, glidants, and lubricants. In some embodiments, the pharmaceutically acceptable excipient(s) are a diluent and a filler. IN some embodiments, the pharmaceutically acceptable excipient(s) are lactose and microcrystalline cellulose. In some embodiments, the diluent is lactose. In some embodiments, the filler is microcrystalline cellulose. In some embodiments, the lubricant is magnesium stearate. In some embodiments, the pharmaceutically acceptable excipient(s) is a diluent, e.g., lactose.

In an embodiment, the at least one pharmaceutically acceptable excipient is lactose and magnesium stearate. In some embodiments, the lactose is present in an amount of about 0% to about 70% by weight, preferably about 5% to about 60% by weight, more preferably, about 10% to about 15% by weight, and most preferably about 14% by weight. In some embodiments, lactose is present in an amount of about 0% to about 40% by weight. In some embodiments lactose is present in an amount of about 2% to about 11% by weight. In some embodiments, the magnesium stearate is present in an amount of about 0.1% to about 5% by weight, preferably about 0.3% to about 3% by weight, more preferably about 0.5% to about 1.5% by weight, and most preferably about 1% by weight. In some embodiments, magnesium stearate is present in an amount of about 0.5% to about 4% by weight.

In certain embodiments, the coating agent is at least one hydrophobic polymer, more preferably at least one hydrophobic, pH-independent polymer. The at least one hydrophobic polymer may be selected from cellulose ethers such as ethyl cellulose, cellulose acetate and the like, polyvinyl esters such as polyvinyl acetate, polyacrylic acid esters, butadiene styrene copolymers, methacrylic and acrylate polymers (pH-independent types), high molecular weight polyvinyl alcohols and waxes such as fatty acids and glycerides, methacrylic acid esters neutral polymer, polyvinyl alcohol-maleic anhydride copolymers and the like, ethylacrylate-methylmethacrylate copolymers, aminoalkyl methacrylate copolymers, and mixtures thereof. In some embodiments, the at least one hydrophobic, pH-independent polymer is ethylcellulose. In some embodiments, the coating agent further comprises a pore forming agent. The coating agent, e.g., the hydrophobic, pH-independent polymer, forms a layer that increases the lag time of active agent release onset relative to an extended-release composition. Preferably the lag time is between 0 to 6 hours, e.g., 1, 2, 3, 4 or 5 hours. In some embodiments, the lag time is between 1 to 6 hours. The coating agent, in some embodiments, decreases the release rate of lamotrigine, e.g., the extent of lamotrigine release, between 6 to 24 hours as compared to an extended-release composition. In some embodiments, a release controlling mechanism is the at least one hydrophobic pH-independent coating agent. The coating agent may be functional or non-functional.

In a manufacturing embodiment, the lamotrigine is blended (mixed) with one or more pharmaceutically acceptable excipients, except the lubricant (e.g., magnesium stearate), prior to the step (a) granulation and the lubricant is blended with the step (a) granules and/or agglomerates.

In a manufacturing embodiment, the granules and/or agglomerates of step (a) are blended with one or more pharmaceutically acceptable excipients in a two step process. In other words, step (b) comprises two sub-steps. In the first sub-step, the granules and/or agglomerates of step (a) are blended with one or more pharmaceutically acceptable excipients, except the lubricant. In the second sub-step, the lubricant is added to the blended excipient/granule/agglomerate mixture.

In an aspect, the controlled-release composition(s) satisfy specific dissolution criteria. In an embodiment, the extended-release composition satisfies the following dissolution criteria, when dissolution takes place using the basket method (USP Apparatus 1) at 50 rpm maintained at 37° C. in 900 mL pre-warmed medium (final pH 6.8) comprising 700 mL 0.01 M HCl and 200 mL phosphate buffer containing sodium lauryl sulfate (pH 12), after two hours, no more than 50% of the lamotrigine is dissolved; during the first 5-hour period of the dissolution, 30-80% of the lamotrigine is dissolved; during the first 10-hour period of the dissolution, at least 60% of the lamotrigine is dissolved. In some embodiments, after two hours, no more than about 50 wt. % of the total amount of the active agent is released. In some embodiments, after five hours, about 30 wt. % to about 80 wt. % of the total amount of the active agent is released. In some embodiments, after ten hours, no less than about 60 wt. % of the total amount of the active agent is released.

In an embodiment, the modified extended-release composition satisfies the following dissolution criteria, when dissolution takes place using the paddle method (USP Apparatus 2) at 50 rpm maintained at 37° C. in 700 mL of 0.01 M HCl (acid phase), after two hours, with an addition of 200 mL pre-warmed phosphate buffer (pH 12) containing sodium lauryl sulfate (final pH of the dissolution media is 6.8), in the first two hours (prior to the addition of the phosphate buffer) no more than 30% of the lamotrigine is dissolved; during the first 7-hour period of the dissolution, 10-60% of the lamotrigine is dissolved; during the first 17-hour period of the dissolution, at least 60% of the lamotrigine is dissolved. In some embodiments, after two hours, no more than about 30 wt. % of the total amount of the active agent is released. In some embodiments, after seven hours, about 10 wt. % to about 60 wt. % of the total amount of the active agent is released. In some embodiments, after seventeen hours, no less than about 60 wt. % of the total amount of the active agent is released.

In some embodiments, a modified extended-release composition is provided that includes: (a) an extended-release core that includes any of the extended-release compositions described herein; and (b) a coating agent. In some embodiments, the coating agent includes at least one hydrophobic polymer, for example, at least one hydrophobic pH-independent polymer. In some embodiments, the at least one hydrophobic polymer comprises, consists of, or consists essentially of ethyl cellulose. In some embodiments, the coating agent further includes at least one pore forming agent.

Other objects, features and advantages of the present invention will become apparent from the following detailed description. It should be understood, however, that the detailed description and specific examples, while indicating preferred embodiments of the invention, are given by way of illustration only, since various changes and modifications within the scope and spirit of the invention will become apparent to one skilled in the art from this detailed description.

### BRIEF DESCRIPTION OF THE DRAWINGS

FIG. 1 illustrates the various formulations provided for herein. FIG. 1A illustrates an extended-release formulation (also referred to herein as a core). FIG. 1B illustrates a modified extended-release formulation. Shown is a core coated with a hydrophobic, pH-independent film. FIG. 1C illustrates a modified extended-release formulation with a release rate modifier present in the coating. Shown is a core coated with a hydrophobic, pH-independent film containing a pore or channel former.

FIG. 2 is a flow diagram showing a manufacturing process that may be used to produce a composition as disclosed herein. Reference is made to Example 1.

FIG. 3 is a graph showing the dissolution profile for a 50 mg lamotrigine extended-release tablets (▪) and modified extended-release tablets (♦) prepared using the methods provided for herein. Reference is made to Example 3.

FIG. 4 is a graph showing the dissolution profile for two extended-release compositions. The graph illustrates that varying the swelling agent, e.g., hydrophilic polymer, content in the extended-release composition affects the active agent, i.e., lamotrigine, release rate. One composition comprises 30% Hypromellose 2208 and 5% Hypromellose 2910 (▪) and the other comprises 25% Hypromellose 2208 and 10% Hypromellose 2910 (▴). Reference is made to Example 4.

FIG. 5 is a graph showing the dissolution profile for a 50 mg lamotrigine modified extended-release tablets having a 4% coat (▴), 9% coat (X), or 11% coat (▪) prepared using the methods provided for herein. Reference is made to Example 4.

### DETAILED DESCRIPTION

The invention will now be described in detail by way of reference only using the following definitions and examples. All patents and publications, including all sequences disclosed within such patents and publications, referred to herein are expressly incorporated by reference.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. It is to be understood that this invention is not limited to the particular methodology, protocols, and reagents described, as these may vary; the materials, methods and examples are illustrative only, and are not intended to be limiting.

The headings provided herein are not limitations of the various aspects or embodiments of the invention which can be had by reference to the specification as a whole. Accordingly, the terms defined immediately below are more fully defined by reference to the specification as a whole.

### DEFINITIONS

The phrase “controlled-release composition” or “controlled release formulation” herein refers to any composition/formulation that comprises lamotrigine and is either an extended-release or modified extended-release composition.

The term “extended-release” herein refers to any composition which comprises lamotrigine, which is formulated to provide a gradual release of lamotrigine over a relatively longer period of time so that the concentration of lamotrigine is maintained in the blood for a longer time at a more uniform concentration than a corresponding immediate release composition comprising the same drug in the same amount. The phrase may be used interchangeably with, for example, delayed release, sustained release, modified release, prolonged release, slow release or pulsed-release at a particular time. “Extended-release pharmaceutical compositions” mean any pharmaceutical composition that is other than immediate release pharmaceutical composition.

The term “modified extended-release” herein refers to an extended-release composition that has at least one additional component or feature that further delays or slows the release of lamotrigine. For example, a hydrophobic, pH-independent coating applied to an extended-release composition will act to further delay the release of the active agent, e.g., lamotrigine, from an extended-release tablets.

The term “matrix” refers to a cross-linked structure formed by the swelling agents in a compressed or compacted dosage form. The cross-linked structure provides a rate controlling mechanism consisting of one or more swelling agent, e.g., hydrophilic polymer, and, optionally, other excipients. Such embodiments will be referred to herein as matrix compositions.

The term “core” as used herein refers to a composition having the active ingredient, e.g., lamotrigine, and at least one swelling agent, and, optionally, other pharmaceutically acceptable excipient(s), wherein the composition has been compressed. A core may be an extended-release composition.

The formulations as described herein deliver a therapeutically effective amount of lamotrigine to a patient for up to 24 hours following a once-daily administration. The term “therapeutically effective amount” is an amount of the active agent which stops or reduces the progress of the condition to be treated or which otherwise completely or partly cures or acts palliatively on the condition. Lamotrigine or a pharmaceutically acceptable salt or derivative thereof may be present from an amount of about 1 mg to about 500 mg in the controlled release formulation. Particularly, the controlled release formulation may comprise lamotrigine or a pharmaceutically acceptable salt or derivative thereof in an amount of 25 mg, 50 mg, 100 mg, 150 mg, 200 mg, 250 mg, 300 mg and/or 400 mg. The recommended dose of Lamictal® XR may also be considered as a standard dose.

The term “lag time” refers to the time required for 20% active agent, e.g., lamotrigine, to be released from the composition being tested.

The phrase “substantially free” when with respect to the aqueous dispersion means that there are no added organic solvents to any commercially sold aqueous dispersion product. Thus, the commercial product may have de minimus quantities of organic solvents that are the result of synthesis or the manufacturing process. It will be understood by one of skill that components used in the formulations described herein may have been produced or synthesized with organic solvents and that residual amounts may be present, i.e., de minimus quantities may be present, that cannot be removed by further processing and may remain even after drying.

The phrase “release rate modifier” refers to a pharmaceutical excipient that, when present in the composition, results in an alteration to the release rate of an active ingredient, e.g., lamotrigine, as compared to the release from an identical composition in which the agent is absent.

A “dosage form” or “dosage formulation” means a unit of administration of an active agent, e.g., lamotrigine. Examples of dosage formulations include tablets, capsules, sachets, mini-tabs, pellets, free flowing granules, beads or pills and the like. “Form” and “formulation” are to be used interchangeably and may be context dependent. The amount of lamotrigine in a formulation as described herein refers to the weight of lamotrigine free base. If a pharmaceutically acceptable lamotrigine salt or derivative is used then the amount of lamotrigine should be adjusted based on its molecular weight.

“Bioavailability” means the extent or rate at which an active agent, e.g., lamotrigine, is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

“Bioequivalence” or “bioequivalent” means the absence of a significant difference in the rate and extent to which the active agent (e.g., lamotrigine) or surrogate marker for the active agent in pharmaceutical equivalents or pharmaceutical alternatives becomes available at the site of action when administered in an appropriately designed study.

The phrase “hydrophobic, pH-independent coating” means any hydrophobic polymer that has a solubility profile that is substantially flat over a pH range of 1-9, preferably 1-7. It is specifically contemplated that the hydrophobic polymer is not an enteric polymer, i.e., soluble at a pH≧4.5, when used alone.

The phrase “swelling agent” as used herein includes without limitation those agents that swell in aqueous media. The swelling agent may also gel. The swelling agent may be one or more swellable polymers, one or more hydrophilic polymer, or one or more swellable compound (e.g., (semi) synthetic polymers), one or more water-insoluble but swellable (upon the presence of water) polymers or compounds or combinations thereof

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, t_{1/2} and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “t_{1/2}” refers to biological half-life: the time required for half the quantity of drug deposited in a living organism to be metabolized or eliminated by normal biological process. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC is the area under the curve of concentration versus time from time 0 to time infinity.

### Active Pharmaceutical Ingredient

The lamotrigine may be present in the final dosage form in an amount of between about 5% w/w to about 70% w/w of the final dosage form. For example, the lamotrigine may be present at any of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% w/w, or any of about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, or about 65% to about 70% w/w of the final dosage form. In one embodiment, the lamotrigine is present in an amount of between about 15% w/w to about 50% w/w of the final dosage form. In another embodiment, the lamotrigine is present in an amount of between about 25% w/w to about 35% w/w or about 30% w/w to about 40% w/w of the final dosage form. In yet another embodiment, the lamotrigine is present in an amount of about 31% w/w of the final dosage form.

In some embodiments, lamotrigine is present at about 6% to about 70% (w/w) in a dosage form (e.g., core), and optionally, a polymer coating (for example, a hydrophobic polymer, e.g., a hydrophobic, pH-independent polymer) adds about 1% to about 5% weight gain to the dosage form. For example, the lamotrigine may be present at any of about 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% w/w, or any of about 6% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, or about 65% to about 70% w/w in a dosage form (e.g., core), and optionally, a polymer coating (for example, a hydrophobic polymer, e.g., a hydrophobic, pH-independent polymer) adds about 1% to about 5% w/w (e.g., about 1%, about 1.5%, about 2%, about 2.5%, about 3%, about 3.5%, about 4%, or about 5% w/w), or about 2% to about 3% w/w weight gain to the dosage form.

The lamotrigine may be present in the final dosage form in an amount of between about 25 mg to about 300 mg, about 25 mg to about 400 mg, or about 25 mg to about 500 mg, wherein the weights are reported as weight of lamotrigine freebase in the dosage form, e.g., tableted dosage form. For example, the lamotrigine may be present in the final dosage form in an amount of any of about 25, 50, 75, 100, 125, 150, 175, 200, 225, 250, 275, 300, 325, 350, 375, 400, 425, 450, 475, or 500 mg. In one embodiment, the lamotrigine may be present in the final dosage form in an amount of about 25 mg. In one embodiment, the lamotrigine may be present in the final dosage form in an amount of about 50 mg. In one embodiment, the lamotrigine may be present in the final dosage form in an amount of about 100 mg. In one embodiment, the lamotrigine may be present in the final dosage form in an amount of about 200 mg. In one embodiment, the lamotrigine may be present in the final dosage form in an amount of about 300 mg. In one embodiment, the lamotrigine may be present in the final dosage form in an amount of about 400 mg. In one embodiment, the lamotrigine may be present in the final dosage form in an amount of about 500 mg.

### Swelling Agents

Suitable swelling agents include swellable polymers, including, but not limited to, hydrophilic polymers and water-insoluble but swellable (upon the presence of water) polymers or compounds. The water-insoluble but swellable polymers or compounds include, but are not limited to, microcrystalline cellulose, sodium carboxymethylcellulose, calcium carboxymethylcellulose, croscarmellose sodium, agar, guar gum, carrageenan, tragacanth, chitosan, alginates and the like and zein.

The term “hydrophilic polymer” as used herein is a material that is a water soluble rate controlling polymer. The hydrophilic polymers possess swellable properties in an aqueous media. The hydrophilic polymers act as a swelling agent. The hydrophilic polymers may be present in the form of a single compound or in the form of a mixture of compounds.

Suitable hydrophilic polymers include, but are not limited to, acrylic acid polymers, such as crosslinked acrylic acid-based polymers; carboxymethylamide, cross-linked polyvinylpyrrolidone, polyvinylacetate, polyvinylalcohols, polyoxyethyleneglycols, non-crosslinked polyvinylpyrrolidone, cellulose derivatives (such as hydroxyethyl cellulose, hydroxypropyl cellulose, hydroxypropyl methylcellulose, methylcellulose, carboxymethyl cellulose and the like), ethylcellulose resins (Ethocel), noncellulose polysaccharides (galactomannans, guar gum, carob gum, gum arabic, sterculia gum, agar, alginates and the like), xanthan gum, casein, zein, gelatin, polyethylene oxide (Polyox resins), sodium carboxymethyl cellulose, vinylpyrrolidone/vinyl acetate copolymer (for example marketed as Plasdone® S-630), polyethylene glycol and the like. The hydrophilic polymers preferably used in the controlled-release compositions are hydroxypropyl methylcelluloses, such as Methocel™ K or E substitution type.

In one aspect the swelling agent functions as a release rate modifier to the lamotrigine core. The amount and specific swelling agent used may be varied to provide a desired release profile of the active ingredient, e.g., lamotrigine.

In an embodiment, the swelling agent is used as a dry powder (i.e., excipient) and blended with the active agent, e.g., lamotrigine, prior to granulation. In some embodiments, the swelling agent is hypromellose, which is a methyl and hydroxypropyl mixed ether of cellulose. Hypromellose contains, calculated on the dried basis, methoxy and hydroxypropoxy groups conforming to the limits for the types of hypromellose (hydroxypropyl methylcellulose) set forth in Table 1 below.

TABLE 1

| | | |
|---|---|---|
| Substitution | Methoxy (%) | Hydroxypropoxy (%) |

| Type | Min. | Max. | Min. | Max. |
|---|---|---|---|---|
| 1828 | 16.5 | 20.0 | 23.0 | 32.0 |
| 2208 | 19.0 | 24.0 | 4.0 | 12.0 |
| 2906 | 27.0 | 30.0 | 4.0 | 7.5 |
| 2910 | 28.0 | 30.0 | 7.0 | 12.0 |

The desired release characteristics of the controlled-release composition(s) is determined by the choice of substitution type and content in the swelling agent. The desired release characteristics may be modified by altering the type and/or combination of hypromellose utilized in the swelling agent.

In one embodiment, the hydrophilic polymer is Hypromellose 2910, USP. In one embodiment, the hydrophilic polymer, e.g., Hypromellose 2910, is present in an amount of about 0% w/w to about 80% w/w, more preferably about 0% w/w to about 70% w/w, even more preferably about 5% w/w to about 50% w/w and most preferably about 5% w/w to about 15% w/w in the core. In some embodiments, the Hypromellose 2910 is present in an amount of about 2% to about 25% w/w in the core. In an embodiment, the Hypromellose 2910 is present in an amount of about 10% w/w in the core. In some embodiments, Hypromellose 2910 may be present in an amount of any of about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67% 68%, 69%, 70%, 71%, 72%, 73%, 74%, 75%, 76%, 77%, 78%, 79%, or 80% w/w, or any of about 0% to about 5%, about 3% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, or about 75% to about 80% w/w in the core.

In one embodiment, the hydrophilic polymer is Hypromellose 2208, USP. In one embodiment, the hydrophilic polymer, e.g., Hypromellose 2208, is present in an amount of about 5% w/w to about 80% w/w, more preferably about 10% w/w to about 70% w/w, even more preferably about 15% w/w to about 50% w/w and most preferably about 20% w/w to about 30% w/w in the core. In some embodiments, the Hypromellose 2208 is present in an amount of about 10% to about 40% w/w in the core. In an embodiment, the Hypromellose 2208 is present in an amount of about 25% w/w in the core. In some embodiments, Hypromellose 2208 may be present in an amount of any of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67% 68%, 69%, 70%, 71%, 72%, 73%, 74%, 75%, 76%, 77%, 78%, 79%, or 80% w/w, or any of about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, or about 75% to about 80% w/w in the core.

In one embodiment, the swelling agent is microcrystalline cellulose. In one embodiment, the swelling agent is a swellable polymer, e.g., microcrystalline cellulose, present in an amount of about 0% w/w to about 70% w/w, more preferably about 5% w/w to about 60% w/w, even more preferably about 15% w/w to about 50% w/w and most preferably about 15% w/w to about 25% w/w in the core. In some embodiments, the microcrystalline cellulose is present in an amount of about 0% to about 60% w/w in the core. In an embodiment, the microcrystalline cellulose is present in an amount of about 19% w/w in the core. In some embodiments, the swellable polymer, e.g., microcrystalline cellulose may be present in an amount of any of about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67% 68%, 69%, or 70% w/w, or any of about 0% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, or about 65% to about 70% w/w in the core.

The swelling agent(s) is/are present (e.g., as a combination of one or more swelling agent(s) such as hypromellose 2208, hypromellose 2910 and/or microcrystalline cellulose) in a total amount of between about 0% w/w to about 80% w/w of the final dosage form. In one embodiment, the swelling agent(s) is/are present in an amount of between about 3% w/w to about 70% w/w of the final dosage form. In another embodiment, the swelling agent (s) is/are present in an amount of between about 5% w/w to about 60% w/w of the final dosage form. In yet another embodiment, the swelling agent (s) is/are present in an amount of about 10% to about 65% w/w of the final dosage form. In yet another embodiment, the swelling agent (s) is/are present in a total amount of about 10-55% w/w of the final dosage form. In an embodiment, the swelling agent(s) is/are present in an amount of about 44% w/w of the final dosage form. In some embodiments, the swelling agent(s) may be present in an amount of any of about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67% 68%, 69%, 70%, 71%, 72%, 73%, 74%, 75%, 76%, 77%, 78%, 79%, or 80% w/w, or any of about 0% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, or about 75% to about 80% w/w in the final dosage form.

The hydrophilic polymer(s), e.g., hypromellose 2208 and/or hypromellose 2910, is/are present (as a combination of one or more hydrophilic polymer) in a total amount of between about 0% w/w to about 80% w/w of the final dosage form. In one embodiment, the hydrophilic polymer(s) is/are present in an amount of between about 3% w/w to about 70% w/w of the final dosage form. In another embodiment, the hydrophilic polymer(s) is/are present in an amount of between about 3% w/w to about 60% w/w of the final dosage form. In yet another embodiment, the hydrophilic polymer(s) is/are present in an amount of about 5% to about 65% w/w of the final dosage form. In an another embodiment, the hydrophilic polymer(s) is/are present in an amount of about 5% to about 55% w/w of the final dosage form. In yet another embodiment, the hydrophilic polymer(s) is/are present in a total amount of about 35% w/w of the final dosage form. In some embodiments, hydrophilic polymer(s) may be present in an amount of any of about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67% 68%, 69%, 70%, 71%, 72%, 73%, 74%, 75%, 76%, 77%, 78%, 79%, or 80% w/w, or any of about 0% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, or about 75% to about 80% w/w in the final dosage form.

In some embodiments, the hydrophilic polymer(s) (e.g., Hypromellose 2208, Hypromellose 2910, and/or microcrystalline cellulose) are present in a dosage form (e.g., in the core) at about 50% to about 80% w/w. For example, the core may contain Hypromellose 2208 at about 25% w/w, Hypromellose 2910 at about 10% w/w, and microcrystalline cellulose at about 15% to about 45% w/w.

Hydrophobic, pH-Independent Polymers

The hydrophobic polymer used is important in controlling the release rate of lamotrigine.

The hydrophobic, pH-independent polymer can be selected from the group consisting of ethyl cellulose, cellulose acetate and the like, polyvinyl esters such as polyvinyl acetate, polyacrylic acid esters, butadiene styrene copolymers, methacrylic and acrylate polymers, high molecular weight polyvinyl alcohols and waxes such as fatty acids and glycerides, methacrylic acid esters neutral polymer, polyvinyl alcohol-maleic anhydride copolymers and the like, ethylacrylate-methylmethacrylate copolymers, aminoalkyl methacrylate copolymers, and mixtures thereof. Suitable hydrophobic, pH-independent polymers for use in the compositions disclosed herein are not limited to the foregoing list.

Ethyl cellulose-Ethyl cellulose aqueous dispersion is most preferably used. Suitable dispersions of ethyl cellulose include those available under the trade names Aquacoat® ECD-30 from FMC Corporation (Philadelphia, USA) and Surelease® from Colorcon (West Point, Pa.). Aquacoat® is an aqueous polymeric dispersion of ethyl cellulose and contains sodium lauryl sulfate and cetyl alcohol while Surelease® is an aqueous polymeric dispersion of ethyl cellulose and contains medium chain triglycerides, oleic acid, ammoniated water and fumed silica.

Ethylacrylate-methylmethacrylate copolymer aqueous dispersion is sold under the trademark Eudragit® NE 30D and Eudragit® RL 30D while Eudragit® RL is a dry powder.

Aminoalkyl methacrylate copolymers, for example, are marketed under the brand name of Eudragit® RS as either a dry powder or an aqueous dispersion.

Without wishing to be bound by theory, it is believed that the hydrophobic, pH-independent polymers create a coat or film structure that will impede the release of the active ingredient, e.g., lamotrigine, from the core (or extended-release dosage form). For example, water slowly permeates the hydrophobic, pH-independent coating when the tablet comes in contact with aqueous fluids causing the core to swell. As the core swells, the hydrophobic, pH-independent coating will begin to crack or fracture without dissolving. Thus, as the swelling agent expands it breaks the coating from the inside out, and, thus, facilitates the release of the lamotrigine from the core. The coating may limit drug release to no more than 30% in the first 2 hours.

In some embodiments, one or more hydrophobic polymer(s) (e.g., ethyl cellulose) is/are present in a composition (e.g., dosage form) in an amount such that it/they provide(s) a coating (e.g., to provide a coated tablet) that adds about 1% to about 5% w/w weight gain to the core (for example, about 1%, about 1.5%, about 2%, about 2.5%, about 3%, about 3.5%, about 4%, or about 5% w/w weight gain). In some embodiments, the hydrophobic polymer(s) add(s) about 2% to about 3% w/w weight gain.

Aqueous Dispersions of Hydrophobic, pH-Independent Polymers

The aqueous dispersions that find use in the present invention can be prepared using well known methods in the art. See, for example, U.S. Pat. Nos. 4,123,403 and 4,502,888; see also Iqbal et al., J. Chem. Soc. Pak. 33(5):634-639 (2011). When the aqueous dispersion is produced de novo, i.e., not a commercial product, it may be produced without organic solvents. Thus, the aqueous dispersion may be free of organic solvents. Or the aqueous dispersion may contain a low level of organic solvents.

Alternatively, a commercial aqueous dispersion of a hydrophobic, pH-independent polymer may be used. Specifically, Eudragit® NE30D, Aquacoat® ECD-30, Surelease® E-7, Eudragit® RS 30D, and/or Eudragit® RL 30D may be used. Specifically contemplated is the use of a commercially available aqueous dispersion that is substantially free of organic solvents. In another embodiment, the aqueous dispersion is free of organic solvents.

The aqueous dispersion of hydrophobic, pH-independent polymer may be used for controlling release rate of lamotrigine from the core and/or extended-release dosage form.

The hydrophobic, pH-independent polymer(s) are used to coat an extended-release formulation producing a modified extended-release formulation. The hydrophobic, pH-independent polymer(s) are applied/sprayed onto the core (e.g., extended-release dosage form) as a coating or film. The modified extended-release dosage form will have a weight increase over the extended-release dosage form, due to the hydrophobic, pH-independent polymer coating, that is from about 1% to about 15%. In other words, a modified extended-release formulation comprising a core (e.g., an extended-release formulation) weighs 101% to 115% of the core. The water insoluble hydrophobic, pH-independent polymer(s) is/are present in an amount that results in an increase in weight of the extended-release core of about 1% w/w to about 15% w/w. Preferably, the hydrophobic, pH-independent polymer is present in an amount selected from 1%, 2%, 3%, 4%, 5%, 6%, 7%, 8%, 9%, 10%, 11%, 12%, 13%, 14%, 15% w/w or any sub-range thereof, e.g., from about 2% to about 7% w/w.

A release rate modifier, e.g., a pore forming excipient, may be added to the coating agent(s) (e.g., rate controlling hydrophobic, pH-independent polymer(s)). Although not wishing to be bound by theory, the pore former may dissolve in an aqueous environment, leaving pores in the polymer coating and allowing release of the drug substance through the pores. In some embodiments, one or more pore forming agent(s) may be present at a total concentration of about 0 to about 30% w/w in the coating or film that decreases release of active agent (lamotrigine). The release rate modifier may be selected from copolyvidone; water soluble polymers such as polyvinylpyrrolidone (PVP); cellulose derived materials, such as hydroxyethyl cellulose, hydroxypropyl methylcellulose, hydroxypropyl cellulose; inorganic salts; sugars; hydroxylated compounds, including polyvinyl alcohols and glycols, such as polyethylene glycol and propylene glycol; methacrylic acid copolymers such as Eudragit® L, S, FS, L30D-55 and L100-55; alginic acid and alginate salts and the like, disintegrants; other miscellaneous agents such as talc and silicon dioxide; gelling agents such as carbomers and xanthum gum; and mixtures of any two or more thereof. It is contemplated that a combination of enteric with non-enteric (e.g., pH-independent) polymers may be used to give a coating that is pH-independent, wherein the enteric polymers function as a release rate modifier.

### Excipients

The conventional excipients according to present invention are those excipients which are commonly used in the art and known to any person skilled in the art. These include, but are not limited to, fillers, diluents, binders, lubricants, flow enhancers, compression enhancers, stabilizers, glidants and the like.

Examples of fillers or diluents include, but are not limited to, corn starch, lactose, sucrose, microcrystalline cellulose, kaolin, mannitol, dextrose, sorbitol, dicalcium phosphate, calcium carbonate, sodium chloride, maltitol, xylitol and the like.

Examples of binders include, but are not limited to methylcellulose, hydroxypropyl cellulose, hydroxyethyl cellulose, hydroxypropyl methylcellulose, sodium carboxymethylcellulose, polyvinylpyrrolidone, sucrose, starch, ethylcellulose, acacia, gelatin, gum arabic, copovidone, polyvinyl alcohol, pullulan, agar, tragacanth, sodium alginate, alginic acid, and the like; glycerides such as for example mono-, di- or triglycerides such as, e.g., stearin, palmitin, laurin, myristin, hydrogenated castor or cottonseed oils, glyceryl palmitostearate, glyceryl behenate and the like; fatty acids and alcohols such as, e.g., stearic, palmitic or lauric acids, stearyl, cetyl or cetosteryl alcohols and the like; and waxes such as for example white wax, bees wax, carnauba wax and the like.

Examples of lubricants and glidants include, but are not limited, to stearates and stearic acid, silicone fluid, talc, waxes, oils, colloidal silicon dioxide, sodium stearyl fumarate, polyethylene glycols, hydrogenated vegetable oil, glyceryl behenate, magnesium trisilicate, microcrystalline wax, yellow beeswax, white beeswax and the like. In some embodiments the stearate is magnesium stearate. In some embodiments, the lubricants are added in a final blending just before preparing a dosage form, e.g., free flowing granules or tablets.

In some embodiments, one or more binder(s)/diluent(s) (e.g., lactose) is/are included in a composition (e.g., dosage form, for example in the core) at about 0% to about 40% w/w, or any of about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% w/w, or any of about 0%, to about 5%, about 5% to about 10%, about 10% to about 15%, about 15% to about 20%, about 20% to about 25%, about 25% to about 30%, about 30% to about 35%, or about 35% to about 40% w/w. In one embodiment, lactose is present in dosage form (e.g., in the core) at about 2% to about 11% w/w.

In some embodiments, one or more lubricant(s)/glidant(s) (e.g., magnesium stearate) is/are included in a composition (e.g., dosage form for example in the core) at about 0.5% to about 4% w/w, or any of about 0.5%, 1%, 1.5%, 2%, 2.5%, 3%, 3.5%, 4%, 4.5%, or 5% w/w, or any of about 0.5% to about 1.5%, about 1% to about 2%, about 1.5% to about 2.5%, about 2% to about 3%, about 2.5% to about 3.5%, about 3% to about 4%, about 3.5% to about 4.5%, or about 4% to about 5% w/w. In one embodiment, magnesium stearate is present in the dosage form (e.g., in the core) at about 1% w/w.

### Methodology

The present invention further relates to processes for manufacturing extended-release and/or modified extended-release pharmaceutical formulations, wherein an aspect is a process comprising:
1) Sifting lamotrigine and all other excipients such as swelling agent(s) (e.g., rate controlling polymer(s)), diluents, etc. (except for the lubricant, e.g., magnesium stearate) through a sieve and mix;
2) Granulating step 1) materials using an aqueous granulating fluid, e.g., water;
3) Removing water, e.g., drying, to form a granulate;
4) Optionally, milling or sieving the dried granulate;
5) Blending the granulate (sieved or unsieved) and a lubricant;
6) Producing dosage forms, e.g., compressing the final lubricated blend into tablets or filling the blend into capsules or sachets; and
7) Optionally, coating tablets or capsules with hydrophobic, pH-independent polymer(s) which may or may not include other coating adjuvants, e.g., plasticizers, anti-tackiness agents, colorants, suspending agents, and/or pore formers.

The present processes for manufacturing pharmaceutical formulations, wherein an aspect is a process comprising:
1) Sifting lamotrigine and all other excipients such as swelling agent(s) (e.g., rate controlling polymer(s)), diluents, etc. (except for the lubricant, e.g., magnesium stearate) through a sieve and mix;
2) Granulating step 1) materials using an aqueous granulating fluid, e.g., water;
3) Removing water, e.g., drying, to form a granulate;
4) Milling or sieving the dried granulate;
5) Blending the sifted (sieved) granulate and a lubricant;
6) Producing dosage forms, e.g., compressing the final lubricated blend into tablets or filling into capsules or sachets; and
7) Optionally, coating tablets or capsules with hydrophobic, pH-independent polymer(s) which may or may not include other coating adjuvants, e.g., plasticizers, anti-tackiness agents, colorants, suspending agents, and/or pore formers.

The granulation step of the above method(s) may provide for the removal of water, e.g., drying, simultaneously with the coating of the lamotrigine, to form the granulate. In such cases, steps 2 and 3 merge into a single step.

In embodiments, the lamotrigine is lamotrigine or a pharmaceutically acceptable salt, solvate, hydrate, derivative, crystalline form or non-crystalline form thereof

As noted in step 7, above, the present compositions may contain a functional coating comprising one or more hydrophobic, pH-independent polymer(s). As used herein, the term “functional coating” denotes a coating that modifies the release properties of the total formulation. In other words, the release profile of a composition with the functional coating is moderately or substantially different from the one without the functional coating.

The present compositions can optionally be coated with a non-functional coating, for example, a cosmetic coating. As used herein, the term “non-functional coating” denotes a coating that is not a functional coating. In other words, the release profile of a composition with the non-functional coating is the same or substantially the same as the one without the non-functional coating. A non-functional coating can have some impact on the release of the active agent due to the initial dissolution of the coating, etc., but would not be considered to be a significant deviation from the non-coated composition.

The granulate may be formed using any method known by one of skill in the art. For example, the granulate can be prepared by wet granulation, melt granulation, extrusion/spheronization, hot fusion and the like, including combinations thereof. In a preferred embodiment, the granulate comprising lamotrigine is prepared by wet granulation. Specifically contemplated is the use of a high shear mixer or a fluid bed granulator.

Lamotrigine particles are combined with swelling agent(s) and other excipients in a high shear mixer to form a dry blend.

The dry blend is agitated and the granulating fluid (e.g., water) is sprayed onto the dry blend. A granulate is obtained as the swelling agent(s) (e.g., hydrophilic polymer(s)) is/are adequately hydrated. Agglomerates may be formed.

The granulate is then dried by any suitable means known to one of skill in the art. Such means include, but are not limited to, spray drying, vacuum drying, oven drying or fluid bed drying. The granulate is dried to a moisture content of about 0.05-5% w/w as measured by weight loss using the loss on drying (LOD) method at 105° C. (U.S. Pharmacopeia <731>Loss on Drying). Preferably the granulates are dried to less than 2.5% w/w water content.

### Dosage Form and Strengths

Once the granulates are blended with the lubricant to form a final dry blend (also may be referred to as a lubricated blend or lubricated granulate), the final dry blend is then (1) compressed into tablets, mini-tabs, pellets, beads or pills, (2) filled into capsules or sachets, or (3) provided as free flowing granules.

Equipment suitable for processing the pharmaceutical formulation of the present invention includes any one or a combination of mechanical sifters, blenders, roller compactors, granulators (high shear mixer, low shear mixer or fluid bed granulator), fluid bed dryers, compression machines, rotating bowls or coating pans, etc. The fluid bed granulators may be either top, side (i.e., tangential) or bottom spray configured.

In various embodiments of the controlled-release compositions provided for herein, the lamotrigine is present in an amount of about 25 mg to about 500 mg, preferably about 25 mg, about 50 mg, about 100 mg, about 200 mg, about 300 mg, or about 400 mg. For example, a dosage form may contain any of about 25, 50, 75, 100, 125, 150, 175, 200, 225, 250, 275, 300, 325, 350, 400, 425, 475, or 500 mg of lamotrigine.

In some embodiments, a controlled-release composition (e.g., a dosage form or the core of a dosage form) contains about 6% to about 70% w/w lamotrigine, about 10% to about 40% w/w Hypromellose 2208, about 2% to about 25% w/w Hypromellose 2910, about 0% to about 60% microcrystalline cellulose, about 0% to about 40% w/w lactose, and about 0.5% to about 4% magnesium stearate. Optionally, a coating of ethyl cellulose (e.g., Surelease E-7-19040 (dry solids)) may be provided in an amount to provide a polymer coat of 1% to about 5% w/w weight gain over the core dosage form.

In some embodiments, a controlled-release composition (e.g., a dosage form or the core of a dosage form) contains about 25 mg to about 300 mg lamotrigine, about 25 mg Hypromellose 2208, about 10 mg Hypromellose 2910, about 60 mg to about 135 mg microcrystalline cellulose, about 12 mg to about 32 mg lactose, and about 3 mg to about 5 mg magnesium stearate. Optionally, about 9 mg to about 10 mg of ethyl cellulose (e.g., Surelease E-7-19040 (dry solids)) may be provided to form a polymer coating.

### Bioequivalency

In one embodiment, bioequivalence is any definition thereof as promulgated by the U.S. Food and Drug Administration or any successor agency thereof. In a specific embodiment, bioequivalence is determined according to the Federal Drug Administration's (FDA) guidelines and criteria, including “Guidance For Industry Bioavailability And Bioequivalence Studies For Orally Administered Drug Products-General Considerations” available from the U.S. Department of Health and Human Services (DHHS), Food and Drug Administration (FDA), Center for Drug Evaluation and Research (CDER) March 2003 Revision 1; and “Guidance For Industry Statistical Approaches To Establishing Bioequivalence” DHHS, FDA, CDER, January 2001, both of which are incorporated herein in their entirety.

In an embodiment, bioequivalence of a composition to a reference drug is determined by an in vivo pharmacokinetic study to determine a pharmacokinetic parameter for the active agent composition. Specifically, bioequivalence can be determined by an in vivo pharmacokinetic study comparing a pharmacokinetic parameter for the two compositions. A pharmacokinetic parameter for the active agent composition or the reference drug can be measured in a single or multiple dose bioequivalence study using a replicate or a nonreplicate design. For example, the pharmacokinetic parameters for active agent composition of the present invention and for a reference drug can be measured in a single dose pharmacokinetic study using a two-period, two-sequence crossover design. Alternately, a four-period, replicate design crossover study may also be used. Single doses of the test composition and reference drug are administered and blood or plasma levels of the active agent are measured over time. Pharmacokinetic parameters characterizing rate and extent of active agent absorption are evaluated statistically.

The area under the plasma concentration-time curve from time zero to the time of measurement of the last quantifiable concentration (AUCₒ₋ₜ) and to infinity (AUC_{0-∞}), Cₘₐₓ, and Tₘₐₓ can be determined according to standard techniques. Statistical analysis of pharmacokinetic data is performed on logarithmic transformed data (e.g., AUCₒ₋ₜ, AUC_{0-∞} or Cₘₐₓ data) using analysis of variance (ANOVA).

Under U.S. FDA guidelines, two products (e.g., an inventive lamotrigine formulation and Lamictal XR® 200 mg) or methods (e.g., dosing under non-fasted versus fasted conditions) are bioequivalent if the 90% Confidence Interval (CI) limits for a ratio of the geometric mean of logarithmic transformed AUC_{0-∞}, AUCₒ₋ₜ, and Cₘₐₓ for the two products or two methods are about 0.80 to about 1.25.

In another embodiment, bioequivalence is determined according to the European Medicines Agency (EMEA) document “Note for Guidance on the Investigation of Bioavailability and Bioequivalence”, issued Jul. 26, 2001, available from EMEA.

To show bioequivalency between two compositions or administration conditions pursuant to Europe's EMEA guidelines, the 90% CI limits for a ratio of the geometric mean of logarithmic transformed AUC_{0-∞} and AUC₀₋ₜ for the two products or methods are about 0.80 to about 1.25. The 90% CI limits for a ratio of the geometric mean of logarithmic transformed Cₘₐₓ for the two products or methods can have a wider acceptance range when justified by safety and efficacy considerations. For example the acceptance range can be about 0.70 to about 1.43, specifically about 0.75 to about 1.33, and more specifically about 0.80 to about 1.25.

In one embodiment, in a given experiment, an active agent composition is considered to be bioequivalent to the reference drug if the Test/Reference ratio for the geometric mean of logarithmic transformed AUC_{0-∞}, AUCₒ₋ₜ, and Cₘₐₓ ratio along with its corresponding lower and upper 90% CI limits are all within a lower limit of about 0.80 and an upper limit of about 1.25. Thus, for direct comparison between an inventive active agent composition and a reference drug, the pharmacokinetic parameters for the active agent composition and the reference drug can be determined side-by-side in the same pharmacokinetic study.

In some embodiments a single dose bioequivalence study is performed under non-fasted or fasted conditions. In an embodiment, a single dose bioequivalence study is performed under non-fasted conditions. In an embodiment, a single dose bioequivalence study is performed under fasted conditions.

In other embodiments, the single dose bioequivalence study is conducted between the active agent composition and the reference drug using the strength specified by the FDA in Approved Drug Products With Therapeutic Equivalence Evaluations (Orange Book).

In some embodiments, an in vivo bioequivalence study is performed to compare all active agent compositions with corresponding strengths of the reference drug (e.g., 50 mg of the active agent). In other embodiments, an in vivo bioequivalence study is performed only for the active agent composition of the present invention at the strength of the reference listed drug product (e.g., the highest approved strength) and at the other lower or higher strengths, the inventive compositions meet an appropriate in vitro dissolution test.

### Methods of Treatment

Methods of treatment with the controlled-release compositions disclosed herein are provided. In some embodiments, a method of treatment is provided that includes administration of a controlled-release composition (e.g., extended-release composition or modified extended-release composition), as described herein, to an individual in need thereof. In some embodiments, the extended-release composition is administered for the treatment of a seizure disorder (e.g., epilepsy). In some embodiments, the extended-release composition is administered once daily. In some embodiments, the extended-release composition is administered once daily in an oral dosage form.

In some embodiments, the controlled-release pharmaceutical composition contains lamotrigine (e.g., lamotrigine, a pharmaceutically acceptable salt, polymorph, solvate, hydrate, or derivative thereof, or combination thereof) at a concentration of about 6% w/w to about 70% w/w, for example, in an extended-release core. Optionally, one or more hydrophobic polymer (e.g., hydrophobic, pH-independent polymer) coats the core in an amount that adds about 1% to about 5% w/w weight gain. In some embodiment, the hydrophobic polymer(s) include ethyl acetate in an amount that adds about 2% to about 3% w/w weight gain. In some embodiments, the coating further includes one or more pore forming agent(s).

In some embodiments, the controlled-release pharmaceutical composition contains one or more swelling agent(s) at a concentration of about 50% w/w to 80% w/w, for example, in the extended-release core. In some embodiments, the swelling agent(s) include Hypromellose 2208 at about 10% to about 40% w/w, Hypromellose 2910 at about 2% to about 25% w/w, and/or microcrystalline cellulose at about 0% to about 60% w/w, for example, in the extended-release core.

In some embodiments, the controlled-release pharmaceutical composition contains one or more binder(s)/diluent(s) (e.g., lactose) at a concentration of about 0% to about 40% w/w, for example, in the extended-release core.

In some embodiments, the controlled-release pharmaceutical compositions contains one or more lubricant(s)/glidant(s) (e.g., magnesium stearate) at a concentration of about 0.5% to about 4% w/w, for example, in the extended-release core.

In some embodiments, the controlled-release pharmaceutical composition contains lamotrigine (e.g., lamotrigine, a pharmaceutically acceptable salt, polymorph, solvate, hydrate, or derivative thereof, or combination thereof) at a concentration of about 6% to about 70% w/w, one or more swelling agent(s) (e.g., Hypromellose 2208 at about 10% to about 40%, Hypromellose 2910 at about 2% to about 25% w/w, microcrystalline cellulose at about 0% to about 60% w/w), one or more binder(s)/diluent(s) (e.g., lactose at about 0% to about 40% w/w), and one or more lubricant(s)/glidant(s) (e.g., magnesium stearate at about 0.5% to about 4%), for example, in an extended-release core. Optionally, one or more hydrophobic polymer(s) (e.g., hydrophobic, pH-independent polymer(s), for example, ethyl cellulose) coat(s) the core in an amount that adds about 1% to about 5% w/w weight gain.

In some embodiments, the extended-release pharmaceutical composition is bioequivalent to a reference drug with a proprietary name of Lamictal XR® when administered to a patient in a fasted or non-fasted state.

In some embodiments, about 25 mg to about 500 mg (e.g., about 25, 50, 75, 100, 125, 150, 175, 200, 225, 250, 275, 300, 325, 350, 375, 400, 425, 450, 475, or 500 mg) of lamotrigine (e.g., e.g., lamotrigine, a pharmaceutically acceptable salt, polymorph, solvate, hydrate, or derivative thereof, or combination thereof) is administered per day (e.g., once daily) in a method of treatment as described herein.

In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); kg (kilograms); μg (micrograms); L (liters); ml (milliliters); μl (microliters); cm (centimeters); mm (millimeters); μm (micrometers); nm (nanometers); ° C. (degrees Centigrade); h (hours); min (minutes); sec (seconds); msec (milliseconds).

### EXAMPLES

The present invention is described in further detail in the following examples which are not in any way intended to limit the scope of the invention as claimed. The attached Figures are meant to be considered as integral parts of the specification and description of the invention. All references cited are herein specifically incorporated by reference for all that is described therein. The following examples are offered to illustrate, but not to limit the claimed invention.

### Example 1

### Method of Manufacture

This example illustrates a process of manufacture of the controlled-release compositions of lamotrigine. See FIG. 1 for a schematic drawing of the components in the dosage form and FIG. 2 for a manufacturing process flow chart.

A flow chart showing an exemplary method of manufacturing controlled-release composition(s) is shown in FIG. 2. The composition contained the following components:

TABLE 2
Lamotrigine Extended Release Tablet, 50 mg

| | |
|---|---|
| Composition | Target |

| Item# | Ingredient | mg/unit | % w/w |
|---|---|---|---|
| 1 | Lamotrigine | 50 | 31 |
| 2 | Hypromellose 2208 | 40 | 25 |
| 3 | Hypromellose 2910 | 16 | 10 |
| 4 | Microcrystalline Cellulose | 30 | 19 |
| 5 | Lactose | 22 | 14 |
| 6 | Magnesium Stearate | 2 | 1 |
| Core tablet | | 160 | 100 |
| 7 | Surelease E-7-19040 (dry solids) | 7 | N/A |

| | | |
|---|---|---|
| Coated tablet | 167 | N/A |

| | |
|---|---|
| Polymer coat weight gain (% w/w) | 4 |

Step 1: Sieve lamotrigine and all other excipients such as diluents, etc. (except the lubricant, e.g., magnesium stearate) through a #20 mesh screen and mix well. This will remove lumps. Charge the mixture into a high shear mixer.
Step 2: Granulating the materials from step 1 using an aqueous granulating fluid, e.g., water.
Step 3: Drying the coated granulate in a fluid bed dryer (e.g., to a moisture content of < or equal to 1% loss on drying).
Step 4: Reducing the particle size of the granulate to achieve a suitable flow for compression by hand sieving or passing through a mill equipped with a suitable screen (e.g., US STD. mesh screen #20);
Step 5: Blending the sifted (sieved) granulate with a lubricant. In this example, magnesium stearate was used as the lubricant. The lubricated sieved granulate may be used as a free-flowing granulate or used to fill capsules or sachets.
Step 6: Compressing the lubricated sieved granulate into a dosage form. In this example, the dosage form was a tablet. The tablets formed are an extended-release composition (FIG. 1A) or a core which can be further processed to form the modified extended-release compositions (FIG. 1B or 1C) described herein; and
Step 7: Coating tablets or capsules with hydrophobic, pH-independent polymer(s), which may (FIG. 1C) or may not (FIG. 1B) include other coating adjuvants, e.g., plasticizers, anti-tackiness agents, colorants, suspending agents, and/or pore formers. The compositions formed are modified extended-release compositions.

The compressed tablets have a hardness of greater than 4 kiloponds (target hardness is dependent on tablet size and strength) and a friability of less than 1%.

The tablets may further be coated with a non-functional coating (e.g., a cosmetic coating) by, for example, spraying onto the tablet (e.g., in perforated pan coater) until a uniform coat is achieved (e.g., a target of no more than 4% percent by weight).

### Example 2

### Dosage Strengths

The following example details varying dosage strengths of controlled-release compositions.

TABLE 3

| | | Strength (mg/unit) | | | | | |
|---|---|---|---|---|---|---|---|
| | | 25 | 50 | 100 | 200 | 300 | |
| Item# | Ingredient | mg/unit | mg/unit | mg/unit | mg/unit | mg/unit | % w/w |
| 1 | Lamotrigine | 25 | 50 | 100 | 200 | 300 | 31 |
| 2 | Hypromellose 2208 | 20 | 40 | 80 | 160 | 240 | 25 |
| 3 | Hypromellose 2910 | 8 | 16 | 32 | 64 | 96 | 10 |
| 4 | Microcrystalline Cellulose | 15 | 30 | 61 | 122 | 182 | 19 |
| 5 | Lactose | 11 | 22 | 44 | 88 | 132 | 14 |
| 6 | Magnesium Stearate | 1 | 2 | 3 | 6 | 10 | 1 |
| Core tablet | | 80 | 160 | 320 | 640 | 960 | 100 |
| 7 | Surelease E-7-19040 (dry solids) | 2 | 5 | 10 | 19 | 29 | N/A |

| | | | | | | |
|---|---|---|---|---|---|---|
| Coated tablet | 82 | 165 | 330 | 659 | 989 | N/A |
| Polymer coat weight gain (% w/w) | 3 | 3 | 3 | 3 | 3 | N/A |

The compositions of Table 3 may be produced without Item #7 to produce extended-release compositions.

### Example 3

### Dissolution

This example illustrates the dissolution profile of tablets (extended-release and modified extended-release) manufactured as in Example 1.

The in vitro specifications for generic products are established based on a dissolution profile. In the case of a generic drug product, the in vitro dissolution profiles are generally the same as or similar to the reference listed drug. In this instance, the reference listed drug is Lamictal XR® 50 mg.

Extended-release tablets (uncoated tablets; see FIG. 1A) were tested for drug release using the basket method (USP Apparatus 1) at 50 rpm maintained at 37° C. in 900 mL pre-warmed medium (final pH 6.8) comprising 700 mL 0.01 M HCl and 200 mL phosphate buffer containing sodium lauryl sulfate (pH 12), using the Basket dissolution method described in U.S. Pharmacopeia <711> Dissolution.

Modified extended-release tablets (coated tablets; see FIG. 1B) were tested for drug release in 700 mL of 0.01 M HCl for 2 hours (acid phase), then after two hours 200 mL pre-warmed phosphate buffer containing sodium lauryl sulfate (pH 12) was added. Final dissolution medium pH was 6.8. Samples were taken 1, 2, 3, 4, 5, 8, 12, 14, and 17 hours (additional samples after 17 hours were taken for information and comparison purposes if needed), and the amount of lamotrigine released determined. The paddle method (USP Apparatus 2) at 50 rpm was employed. The temperature of the dissolution media was maintained at 37° C. Dissolution tests of core tablets were performed as described above, i.e., without the acid phase and using basket method (to prevent sticking), for comparison.

FIG. 3 shows the dissolution profile of 50 mg lamotrigine tablets. The extended-release tablet (▪) shows a more rapid release of lamotrigine than does the modified extended-release tablet (♦).

The modified extended-release tablet's dissolution profile was similar to and comparable with Lamictal XR® (data not shown).

### Example 4

### Effects of Swelling Agent and/or Coating Agent

This example illustrates the effect of altering either the swelling agent or coating agent on the dissolution profile of 50 mg tablets (extended-release or modified extended-release) manufactured as in Example 1.

The extended-release compositions tested were produced using the hypromellose mixtures provided in Table 4. As shown in FIG. 4 altering the proportion of the two hypromellose ingredients modifies the lamotrigine release characteristics. Selection and/or combination of other hypromellose products can “fine tune” the drug release characteristics.

TABLE 4

| Lot# | Hypromellose 2208 | Hypromellose 2910 |
|---|---|---|
| 30-5 | 30% | 5% |
| 25-10 | 25% | 10% |

In a second experiment, a 50 mg extended-release tablet having a 30-5 core (as described in Table 4) was coated with a coating agent, Surelease®, according to the manufacturer's instructions, to various levels. The addition of the polymer coat was applied in an amount that results in a weight gain (w/w) of 4%, 9% or 11% of the extended-release tablet. No pore forming agent was added to the commercially available polymer product.

As shown in FIG. 5 increasing the coating level results in an increased lag time and a decrease in the rate of drug release. Thus, the coating level modifies the drug release characteristics.

It will be apparent that combining the choice of swelling agent with the coating agent (type or level) allows for the modification of drug release characteristics.

### Example 5

### In Vivo Pharmacokinetics

This example describes the in vivo pharmacokinetics after a single oral dose of a modified extended-release composition described herein.

After a single dose oral administration to human subjects, the disclosed controlled-release dosage form containing 50 mg lamotrigine provides pharmacokinetics within the following ranges (from analyzing its plasma concentrations):

TABLE 5

| Subject condition | Fasting | Fed |
|---|---|---|
| AUC_{0-∞} (ng/mL × hr) | 20000-40500 | 20000-40500 |
| Cₘₐₓ (ng/mL) | 320-600 | 320-600 |
| tₘₐₓ (hr) | 10-30 | 10-30 |

A 2-arm, open-label, single-dose, fasted relative bioavailability study of the lamotrigine extended release tablets as described in Example 1 versus Lamictal XR® 50 mg tablet reference (“Reference”) is performed in 12 healthy, adult subjects. Each subject participates in two dosing periods separated by a washout period of 7 days. Lamotrigine plasma concentrations in the blood samples are measured and compared.

The lamotrigine concentration-time data are used to calculate the following pharmacokinetic parameters: AUCₒ₋ₜ, AUC_{0-∞}, Cₘₐₓ, Tₘₐₓ and t_{1/2}. The pharmacokinetic parameters are evaluated statistically by an analysis of variance (ANOVA). Analyses of AUC₀₋ₜ, AUC_{0-∞} and Cₘₐₓ are performed on Ln-transformed data.

### Example 6

### Dosage Strengths

The following example details varying dosage strengths of controlled-release compositions, while keeping the tablet sizes similar across all strengths.

TABLE 6

| | | Strength (mg/unit) | | | | |
|---|---|---|---|---|---|---|
| | | 25 | 50 | 100 | 200 | 300 |
| Item# | Ingredient | mg/unit | mg/unit | mg/unit | mg/unit | mg/unit |
| 1 | Lamotrigine | 25 | 50 | 100 | 200 | 300 |
| 2 | Hypromellose 2208 | 75 | 75 | 75 | 75 | 75 |
| 3 | Hypromellose 2910 | 30 | 30 | 30 | 30 | 30 |
| 4 | Microcrystalline Cellulose | 135 | 110 | 60 | 69 | 78 |
| 5 | Lactose | 32 | 32 | 32 | 22 | 12 |
| 6 | Magnesium Stearate | 3 | 3 | 3 | 4 | 5 |
| Core tablet | | 300 | 300 | 300 | 400 | 500 |
| 7 | Surelease E-7-19040 (dry solids) | 9 | 9 | 9 | 10 | 10 |

| | | | | | |
|---|---|---|---|---|---|
| Coated tablet | 309 | 309 | 309 | 410 | 510 |
| Polymer coat weight gain (% w/w) | 3 | 3 | 3 | 2.5 | 2 |

The compositions of Table 6 may be produced without item #7 to produce extended-release compositions.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entireties for all purposes.

### CITATION LIST

### Patent Literature

### WO 2004/012741

US. Pat. Pub. No. 2004/0192690
US. Pat. Pub. No. 2005/0032799
US. Pat. Pub. No. 2009/0022789
US. Pat. Pub. No. 2009/0196924
US. Pat. Pub. No. 2010/0297195
US. Pat. Pub. No. 2011/0052686

What is claimed is:

1. A modified extended-release composition comprising:
(a) an extended-release core comprising:
(i) lamotrigine;
(ii) a swelling agent; and
(iii) optionally, one or more pharmaceutically acceptable excipient; and
(b) a coating agent comprising:
(i) a hydrophobic, pH-independent polymer; and
(ii) optionally, a pore forming agent.

2. An extended-release composition comprising lamotrigine, and at least one swelling agent.

3. The extended-release composition of claim 2 further comprising at least one pharmaceutically acceptable excipient.

4. The extended-release composition of claim 2, wherein the lamotrigine is present in an amount of about 25 mg to about 400 mg.

5. The extended-release composition of claim 2, wherein the lamotrigine content is from about 5% to about 70% by weight.

6. The extended-release composition according to claim 2, wherein the at least one swelling agent is a hydrophilic polymer or a mixture of hydrophilic polymers.

7. The extended-release composition according to claim 2, wherein the at least one swelling agent is selected from Hypromellose, microcrystalline cellulose, carboxymethylamide, cross-linked polyvinylpyrrolidone, polyvinylacetate, polyvinylalcohols, polyoxyethyleneglycols, non-cross-linked polyvinylpyrrolidone, cellulose derivatives, ethylcellulose resins, noncellulose polysaccharides, galactomannans, guar gum, carob gum, gum arabic, sterculia gum, agar, alginates, xanthan gum, casein, zein, gelatin, polyethylene oxide, sodium carboxymethyl cellulose, vinylpyrrolidone/vinyl acetate copolymer, and polyethylene glycol.

8. The extended-release composition according to claim 2, wherein the at least one swelling agent is a mixture of Hypromellose 2208, Hypromellose 2910 and microcrystalline cellulose.

9. The extended-release composition according to claim 8, wherein the Hypromellose 2208 is present in an amount of about 5% to about 80% by weight.

10. The extended-release composition according to claim 9, wherein the Hypromellose 2208 is present in an amount of about 15% to about 50% by weight.

11. The extended-release composition according to claim 8, wherein the Hypromellose 2910 is present in an amount of about 0% to about 80% by weight.

12. The extended-release composition according to claim 11, wherein the Hypromellose 2910 is present in an amount of about 5% to about 15% by weight.

13. The extended-release composition according to claim 8, wherein the microcrystalline cellulose is present in an amount of about 0% to about 70% by weight.

14. The extended-release composition according to claim 13, wherein the microcrystalline cellulose is present in an amount of about 5% to about 60% by weight.

15. The extended-release composition according to claim 3, wherein the at least one pharmaceutically acceptable excipient is selected from diluents, fillers, binders, flow enhancers, compression enhancers, stabilizers, glidants, and lubricants.

16. The extended-release composition according to claim 15, wherein the at least one pharmaceutically acceptable excipient is lactose and magnesium stearate.

17. The extended-release composition according to claim 16, wherein the lactose is present in an amount of about 0% to about 70% by weight.

18. The extended-release composition according to claim 17, wherein the magnesium stearate is present in an amount of about 0.1% to about 5% by weight.

19. An extended-release composition comprising about 5% to about 70% by weight of lamotrigine, from about 5% to about 80% Hypromellose 2208 by weight, from about 0% to about 80% Hypromellose 2910 by weight, from about 0% to about 70% lactose by weight, from about 0% to about 70% microcrystalline cellulose by weight, and from about 0.1% to about 5% magnesium stearate by weight.

20. The extended-release composition according to claim 2, wherein the at least one swelling agent forms a matrix.

21. The composition according to claim 1, wherein said composition is in a final dosage form selected from tablets, capsules, pills, mini-tabs, sprinkles, pellets, or beads.

22. The composition according to claim 2, wherein said composition is in a final dosage form selected from tablets, capsules, pills, mini-tabs, sprinkles, pellets, or beads.

23. A modified extended-release composition comprising an extended-release composition according to claim 2, further comprising a coating agent.

24. A modified extended-release composition according to claim 23, wherein the coating agent is at least one hydrophobic polymer.

25. A modified extended-release composition according to claim 23, wherein the coating agent is at least one hydrophobic, pH-independent polymer.

26. The modified extended-release composition of claim 25, wherein the hydrophobic, pH-independent polymer forms a layer that modifies the release profile of the lamotrigine from the core as compared to the core without the hydrophobic, pH-independent coating agent.

27. A modified extended-release composition according to claim 23, wherein the at least one hydrophobic, pH-independent polymer is selected from cellulose ethers, ethyl cellulose, cellulose acetate, polyvinyl esters, polyvinyl acetate, polyacrylic acid esters, butadiene styrene copolymers, methacrylic and acrylate polymers, high molecular weight polyvinyl alcohols and waxes, fatty acids, glycerides, methacrylic acid esters neutral polymer, polyvinyl alcohol-maleic anhydride copolymers, ethylacrylate-methylmethacrylate copolymers, aminoalkyl methacrylate copolymers, and mixtures thereof.

28. A modified extended-release composition according to claim 27, wherein the at least one hydrophobic, pH-independent polymer is ethylcellulose.

29. A modified extended-release composition according to claim 28, wherein the coating agent further comprises a pore forming agent.

30. The modified extended-release composition according to claim 29, wherein a release controlling mechanism is the at least one hydrophobic, pH-independent coating agent.

31. The modified extended-release composition of claim 25, wherein the hydrophobic, pH-independent polymer forms a layer that increases the lag time of active agent release onset relative to an extended-release composition.

32. The modified extended-release composition of claim 31, wherein the lag time is between 0 to 6 hours.

33. The modified extended-release composition of claim 23, wherein the coating agent decreases the release rate of lamotrigine between 6 to 24 hours as compared to an extended-release composition.

34. The extended-release composition of any of claim 2, that satisfies the following dissolution criteria, when dissolution takes place using the basket method (USP Apparatus 1) at 50 rpm maintained at 37° C. in 900 mL pre-warmed medium (final pH 6.8) comprising 700 mL 0.01 M HCl and 200 mL phosphate buffer containing sodium lauryl sulfate (pH 12), after two hours, no more than 50% of the lamotrigine is dissolved; during the first 5-hour period of the dissolution, 30-80% of the lamotrigine is dissolved; during the first 10-hour period of the dissolution, at least 60% of the lamotrigine is dissolved.

35. The modified extended-release composition of any of claim 23 that satisfies the following dissolution criteria, when dissolution takes place using the paddle method (USP Apparatus 2) at 50 rpm maintained at 37° C. in 700 mL of 0.01 M HCl (acid phase), after two hours, with an addition of 200 mL pre-warmed phosphate buffer (pH 12) containing sodium lauryl sulfate (final pH of the dissolution media is 6.8), in the first two hours (prior to the addition of the phosphate buffer) no more than 30% of the lamotrigine is dissolved; during the first 7-hour period of the dissolution, 10-60% of the lamotrigine is dissolved; during the first 17-hour period of the dissolution, at least 60% of the lamotrigine is dissolved.

36. A method of manufacturing a composition, said method comprising the steps:
(a) granulating lamotrigine, one or more pharmaceutically acceptable excipients, and one or more hydrophilic polymers to form granules and/or agglomerates;
(b) blending the granules and/or agglomerates of step (a) with a lubricant to form a dry blend; and
(c) preparing a dosage form from the dry blend.

37. The method according to claim 36, wherein the at least one pharmaceutically acceptable excipient is selected from diluents, fillers, binders, flow enhancers, compression enhancers, stabilizers, pore formers, pH modifiers, and glidants.

38. The method according to claim 37, wherein the lubricant is magnesium stearate.

39. The method according to claim 37, wherein the one or more pharmaceutically acceptable excipients is a diluent.

40. The method according to claim 39, wherein the diluent is lactose.

41. The method according to claim 36, further comprising coating the dosage form with a coating agent.

42. The method according to claim 41, wherein the coating agent is a hydrophobic, pH-independent polymer.

43. The method according to claim 41, wherein the coating agent further comprises a pore former.

44. A method of manufacturing a composition, said method comprising the steps:
(a) granulating lamotrigine and one or more hydrophilic polymers to form granules and/or agglomerates;
(b) blending the granules and/or agglomerates of step (a) with one or more pharmaceutically acceptable excipients to form a dry blend;
(c) blending a lubricant with the dry blend to form a lubricated dry blend; and
(d) preparing a dosage form from the lubricated dry blend.

45. The method according to claim 44, wherein the at least one pharmaceutically acceptable excipient is selected from diluents, fillers, binders, flow enhancers, compression enhancers, stabilizers, pore formers, pH modifiers, and glidants.

46. The method according to claim 45, wherein the lubricant is magnesium stearate.

47. The method according to claim 45, wherein the one or more pharmaceutically acceptable excipients is a diluent.

48. The method according to claim 47, wherein the diluent is lactose.

49. The method according to claim 44, further comprising coating the dosage form with a coating agent.

50. The method according to claim 49, wherein the coating agent is a hydrophobic, pH-independent polymer.

51. The method according to claim 49, wherein the coating agent further comprises a pore former.
